(19) United States
(12) Patent Application Publication
(10) Pub. No.: US 2013/0133650 A1
(43) Pub. Date: May 30, 2013

(54) GAS-BASED TREATMENT FOR INFECTIVE DISEASE

(71) Applicants: Xilin Zhao, Livingston, NJ (US); Karl Drlica, New York, NY (US)

(72) Inventors: Xilin Zhao, Livingston, NJ (US); Karl Drlica, New York, NY (US)

(73) Assignee: UNIVERSITY OF MEDICINE AND DENTISTRY OF NEW JERSEY, Somerset, NJ (US)

(21) Appl. No.: 13/813,988

(22) PCT Filed: Aug. 2, 2011
(86) PCT No.: PCT/US2011/046337

§ 371 Date: Feb. 4, 2013

Related U.S. Application Data
(60) Provisional application No. 61/369,874, filed on Aug. 2, 2010.

(51) Int. Cl. A61K 33/00; A61M 16/12

(52) U.S. Cl. CPC A61K 33/00; A61M 16/12
USPC 128/203.12; 424/600; 424/700; 423/648.1

(57) ABSTRACT

A gas mixture for treatment of a mycobacterial infection and methods thereof, wherein the gas mixture comprises hydrogen. In certain applications, the gas mixture further comprises oxygen and optionally an inert or anaerobic gas, preferably selected from the group consisting of nitrogen, helium, argon, carbon dioxide, and mixtures thereof. The methods for treatment comprise direct inhalation of the gas mixture comprising hydrogen and oxygen, intubation of a patient with a double lumen endotracheal tube thereby supplying one lung with an anaerobic gas, and administration of a gas mixture comprising hydrogen and oxygen in a hyperbaric setting. Also provided is a method of sterilization of a mycobacterium-contaminated surface comprising administration of the hydrogen-containing gas mixture.

### CROSS-REFERENCE TO RELATED APPLICATIONS

This application claims priority under 35 U.S.C. §119(e) to U.S. Application No. 61/369,874, filed on Aug. 2, 2010, the disclosure of which is incorporated herein by reference in its entirety.

### STATEMENT REGARDING FEDERALLY FUNDED RESEARCH

This invention was made with government support under Grant NIH DP2-OD007423 and R01 A1073491 awarded by the National Institutes of Health. Accordingly, the U.S. Government has certain rights in this invention.

### FIELD OF THE INVENTION

This invention relates to a method of gas-based bacterial killing that can be used to treat infectious disease, more particularly to a novel method for tuberculosis, therapy.

### BACKGROUND OF THE INVENTION

Mycobacterium is a genus of bacterium including Mycobacterium tuberculosis and Mycobacterium bovis. Mycobacteria can colonize their hosts without the hosts showing any adverse signs. For example, billions of people around the world have asymptomatic infections of M. tuberculosis. Mycobateria can also infect a wide range of species, including non-human primates, elephants and other exotic ungulates, carnivores, marine mammals and psittacine birds. Montali, R. J., 2001 Rev Sci Tech. 20(1):291-303. Mycobacterial infections are notoriously difficult to treat. The organisms are hardy due to their cell wall, which is neither truly Gram negative nor positive. Additionally, they are naturally resistant to a number of antibiotics that disrupt cell-wall biosynthesis, such as penicillin. Due to their unique cell wall, they can survive long exposure to acids, alkalis, detergents, oxidative bursts, lysis by complement, and many antibiotics. Most mycobacteria are susceptible to antibiotics, such as rifamycin, but antibiotic-resistant strains have emerged. As with other bacterial pathogens, surface and secreted proteins of M. tuberculosis contribute significantly to the virulence of this organism.

Mycobacterium tuberculosis, the causative agent of tuberculosis, infects a third of world's human population and kills 1.7 million persons a year. Since impaired immune function allows latent tuberculosis to become active, the spread of HIV-1/AIDS, increased use of immunosuppressant chemicals for autoimmune disease and organ transplantation, and use of radio/chemotherapy for cancer patients are contributing to a global tuberculosis problem. Effective anti-tuberculosis chemotherapies exist, but the requirement for long treatment periods with multiple agents can lead to patient compliance and drug supply difficulties that cause treatment to be sporadic. In particular, chemotherapy of tuberculosis requires long treatment periods in which logistical problems and adverse reactions make it difficult for patients to adhere to therapy. Treatment is often administered on an outpatient basis, and is given for six to nine months, although it may be administered for years in some cases due to a patient's lack of compliance and inability to take the drugs prescribed. The need for long treatment periods is also attributed in part to a fraction of the infecting bacteria entering a dormant (persistent) state in which antimicrobial susceptibility is thought to diminish. Poor patient compliance also contributes to the selective amplification of resistant bacterial subpopulations and to the emergence of multidrug-resistant strains of Mycobacterium tuberculosis.

These factors, plus high bacterial burden in pulmonary tuberculosis, contribute to an increasing prevalence of multidrug-resistant (MDR) tuberculosis, which is now estimated to represent 5% of the cases globally. Extensively drug-resistant (XDR) tuberculosis has been reported from many countries, and in some localities it can represent more than 20% of the cases. Moreover, cases in which bacilli become resistant to all available drugs (completely drug resistant (CDR) tuberculosis) are emerging in many countries. The key requirements for sustainable tuberculosis control (or eventual eradication of the disease) include shortening treatment time, preventing new drug-resistance, overcoming drug-resistance that has already developed, and effective killing of both growing and dormant (growth-arrested) bacilli.

The physiology of M. tuberculosis is highly aerobic and requires high levels of oxygen. Primarily a pathogen of the mammalian respiratory system, M. tuberculosis infects the lungs. Its unusual cell wall, which is rich in lipids (e.g., mycolic acid), is likely responsible for its resistance and is a key virulence factor. M. tuberculosis has a complex relationship with oxygen. Removal of oxygen by transfer of cultures to an anaerobic jar leads to death of the bacilli with a half-life of 10 hours. Wayne, L. and Lin, K., 1982 Infect. Immun. 37:1042-1049. But when oxygen is removed very slowly, over the course of two weeks, M. tuberculosis enters a non-replicative, persistent state. In this state the bacteria become dormant and are tolerant to anaerobiosis and many anti-tuberculosis agents. Wayne, L. G. and Hayes. L. G., 1996 Infect. Immun. 64:2062-2069. These in vitro observations help explain the effectiveness of collapse therapy, an approach that predates anti-tuberculosis chemotherapy. In collapse therapy, air is expelled from an infected lung through artificial pneumothorax, pneumoperitoneum, or implantation of plombage. Due to the passive and gradual nature of oxygen depletion in infected areas of lungs, collapse therapy may convert tubercle bacilli from an actively growing phase into a non-replicating, persistent (dormant) state. Consequently, these procedures are expected to be bacteriostatic rather than bactericidal.

More recently, an in vitro model was reported involving growth arrest of Mycobacterium bovis BCG, a close relative of M. tuberculosis, with diethylene-triamine-nitric oxide adduct (DETA-NO), a generator of nitric oxide. Hussain, Syed et al., January 2009 Antimicrob. Agents and Chemother. 157-161. Growth arrest of M. bovis BCG was sustained for 72 hours with a single treatment of DETA-NO. However, exposure to air reinstated growth. It was also reported that anaerobic shock caused cell death that was not blocked by pretreatment with DETA-NO.

Applicants have recognized that none of the current approaches for tuberculosis intervention, including promising new drugs under development, meet the key requirements for sustainable tuberculosis control discussed above. Finding alternative approaches for rapid and effective tuberculosis therapy is therefore a public health priority. The present invention addresses these needs, among others.

### SUMMARY OF THE INVENTION

Provided herein is a gas mixture for treatment of a mycobacterial infection comprising hydrogen. In certain embodiments, the gas mixture further comprises oxygen having a partial pressure of from about 0.17 to about 0.30, resulting in a breathable, aerobic gas mixture. In certain other embodiments, the gas mixture further comprises an anaerobic gas, preferably an anaerobic gas selected from the group consisting of nitrogen, helium, argon, carbon dioxide, and mixtures thereof. In certain embodiments, the gas mixture at about one atmosphere of pressure comprises hydrogen in an amount of from about 0.1% to about 85% by volume, preferably of from about 1.0% to about 83% by volume, and more preferably of from about 2.5% to about 80% by volume. In certain embodiments, the gas mixture at a pressure of about one atmosphere comprises hydrogen in an amount outside of explosion limits, as is readily apparent to one of ordinary skill in the art, and preferably in an amount offrom about 2.5% to about 3.5% by volume or about 78% to about 80% by volume.

Also provided herein is a method for treatment of a mycobacterial respiratory tract infection in a patient comprising administering a gas mixture comprising hydrogen and oxygen to the respiratory tract of the patient via direct inhalation under at a pressure of about one atmosphere. In certain embodiments, the mycobacterial infection is a respiratory tract infection due to the presence of M tuberculosis or M Bovis. In certain embodiments, the gas mixture further comprises an inert gas, preferably selected from the group consisting of nitrogen, helium, argon, and mixtures thereof. In certain other embodiments, the step of administering the gas mixture into the respiratory tract of the patient is carried out at a pressure of about one atmosphere, and the gas mixture comprises hydrogen in an amount of from about 0.1% to about 4% by volume or about 75% to about 85% by volume, preferably of from about 1.0% to about 3.8% by volume or about 76% to about 83% by volume, and more preferably of from about 2.5% to about 3.5% by volume or about 78% to about 80% by volume. In certain embodiments, the gas mixture comprises oxygen in an amount of from about 15% to about 50% by volume, preferably of from about 17% to about 40% by volume, and more preferably of from about 20% to about 25% by volume.

Also featured herein is a method for treatment of a mycobacterial respiratory tract infection in a patient comprising (a) intubating the patient with a double lumen endotracheal tube, (b) ventilating a first lung containing the mycobacterial infection with a gas mixture comprising an anaerobic gas, and (c) ventilating a second lung with air or oxygen. In certain embodiments, the anaerobic gas comprises hydrogen. In certain embodiments, the anaerobic gas is selected from the group consisting of nitrogen, argon, helium, carbon dioxide, and mixtures thereof. In certain other embodiments, after sufficient time to kill most of the bacteria in the first lung, the gas connection to the two lumens is switched such that the second lung receives the gas mixture and the first lung receives air or oxygen. In this way both lungs are treated. In certain preferred embodiments, the gas mixture at a pressure of about one atmosphere comprises hydrogen in an amount of about 10% by volume, nitrogen in amount of about 85% by volume, and carbon dioxide in an amount of about 5% by volume. In certain embodiments, the gas mixture at a pressure of about one atmosphere comprises nitrogen in amount of about 40% by volume, and argon in an amount of about 40% by volume, and helium in an amount of about 20% by volume.

Also provided herein is a method for treatment of a mycobacterial respiratory tract infection in a patient comprising (a) enclosing the patient in a hyperbaric chamber, (b) filling the hyperbaric chamber to a pressure of from about 3.5 to about 50 atmospheres with a gas mixture comprising hydrogen and oxygen, wherein the oxygen has a partial pressure of about 0.17 to about 0.30, and (c) administering the gas mixture to the respiratory tract of the patient via direct inhalation of the gas mixture. In certain embodiments, the gas mixture further comprises an inert gas selected from the group consisting of nitrogen, helium, argon, and mixtures thereof as balance gas to hydrogen and oxygen. In certain preferred embodiments, the pressure in the hyperbaric chamber is from about 4 to about 10 atmospheres.

Also featured herein is a method for the sterilization of mycobacterial-contaminated surface comprising exposing the contaminated surface to a gas mixture comprising hydrogen. In certain embodiments, the surface is the skin of a patient having a mycobaterial infection of the skin or body extremities. In certain embodiments, the surface is equipment used for clinical and experimental research applications.

### BRIEF DESCRIPTION OF THE FIGURES

FIG. 1 illustrates the effect of gases and gas mixtures on M. tuberculosis survival; exponentially growing cultures of M. tuberculosis strain H37Rv were treated with gases and gas mixtures comprising: (A) compressed air (filled triangles), carbon dioxide (open triangles), nitrogen (filled circles), and Bioblend (open circles); and (B) helium (filled circles), helium-modified Bioblend (nitrogen/helium/carbon dioxide at a ratio of 85/10/5%, filled squares), argon (open triangles), NAH (nitrogen/argon/helium at a ratio of 40/40/20%, filled triangles), and hydrogen (open squares).

FIG. 2 illustrates the effect of Bioblend shock on survival of M. tuberculosis strains differing in drug susceptibility and physiological status; (A) Bioblend-mediated killing of clinical isolates having various drug-resistance profiles (TN 10775 (a drug pan-sensitive isolate, diagonal bars), TN 10536 (an isoniazid-resistant isolate, white bars), TN 1626 (an MDR isolate, horizontal bars), and KD505 (an XDR isolate, solid bars)); (B) Bioblend treatment of homogenate from rabbit lung infected with M. tuberculosis strain HN878 (diagonal bars: right lung, 4 weeks after infection (exponentially growing phase); white bars: left lung, 8 weeks after infection (growth-arrest (dormant) phase); solid bars: right lung, 8 weeks after infection (growth-arrest (dormant) phase)); (C) comparison of Bioblend-mediated killing of growing and dormant M. tuberculosis (M. tuberculosis strain H37Rv samples were treated with Bioblend and processed as in FIG. 2(A) when growing aerobically (diagonal bars) or when growth was arrested by gradual oxygen depletion (20 days of sealed tube growth, horizontal bars)).

FIG. 3 illustrates the effect of anaerobic shock on survival of M. tuberculosis inside human macrophage-like cells; (A) Bioblend-mediated killing of M. tuberculosis. Bioblend (diagonal bars) and argon (horizontal bars); (B) Bioblend-mediated cytotoxicity with uninfected THP-1 macrophage-like cells (THP-1 cells were treated with Bioblend (diagonal bars), argon (horizontal bars), or compressed air (solid bars) for the indicated times).

FIG. 4 illustrates the effect of hydrogen-oxygen mixtures on M. tuberculosis strain H37Rv survival after treatment with hydrogenized air (3.2% hydrogen, balance (96.8%) air; squares) or oxygenized hydrogen (1.5% oxygen, balance (98.5%) hydrogen; circles) for the indicated times as described in Methods.

FIG. 5 illustrates the effect of gas treatment on survival of growing M. bovis BCG that were serially diluted and applied on 7H10 agar plates placed into anaerobic jars after which the jars were flushed with helium (triangles), Bioblend (squares) or hydrogen (circles) for the indicated times before the plates were taken out of the jars for recovery growth of the bacteria.

### DETAILED DESCRIPTION OF THE INVENTION

The present invention relates to gas compositions and methods of use thereof to treat infectious diseases, particularly those diseases for which the infecting agent is present in the respiratory tract. In certain embodiments, the infectious disease is caused by a member of the Mycobacterium genus, and preferably an infection caused by M tuberculosis. While mycobacteria do not seem to fit the Gram-positive category from an empirical standpoint (i.e., they generally do not retain the crystal violet stain well), they are classified as an acid-fast Gram-positive bacterium due to their lack of an outer cell membrane. All Mycobacterium species share a characteristic cell wall, thicker than in many other bacteria, which is hydrophobic, waxy, and rich in mycolic acids/mycolates. Accordingly, one skilled in the art would understand that the present invention and method of treatment described herein applies to the treatment of infections caused by all Mycobacterium species, including, but not limited to, M tuberculosis, M. bovis and M. leprae.

The present invention also provides a method of treatment of a mycobacterial infection in a patient. As used herein, the term “patient” is used to mean an animal; including, but not limited to a mammal, including a human, non-human primates, and elephants. In particular, the present invention demonstrates efficacy with cultured Mycobacterium tuberculosis, the causative agent of human tuberculosis. The key requirements for sustainable control and eventual eradication of tuberculosis are shortening treatment time, preventing new drug-resistance from emerging, overcoming drug-resistance that has already developed, and eradicating both growing and growth-arrested tubercle bacilli. Treatment of infected lungs with anaerobic gas, and in particular hydrogen or hydrogen-containing gas satisfies these criteria.

The gas-based treatment can be widely used for all forms of pulmonary tuberculosis. Gas treatment may rapidly eradicate M. tuberculosis infection if the treatment gas reaches all foci of the infected lung. Even if the gases used are unable to penetrate granulomas that are far from airways, which is less likely to be the case for a small gas molecule under high pressure in a hyperbaric setting, gas-mediated treatment will still act to convert a patient from an open-lesion, contagious disease state to a non-contagious stage in hours, if not minutes. Achieving a similar goal with traditional multi-drug combination therapy requires months.

The gas shock approach is especially useful for treatment of multidrug resistant (MDR)-, extensively drug resistant (XDR)-, and completely drug resistant (CDR)-tuberculosis, since traditional chemotherapy is at best marginally effective with these forms of tuberculosis. Anaerobic or hydrogen gas treatment is also useful for cases deemed unsuitable for surgical interventions, such as bilateral, multi-foci, or heavily infiltrated lesions.

In principle, gas-based therapy meets four key requirements for tuberculosis control: treatments are expected to be short, to rarely select new resistant mutants, to overcome existing drug resistance, and to effectively kill both growing and non-growing (dormant) cells. No mutant resistant to Bioblend shock has been detected (few are expected, since the shock kills so rapidly and extensively). Selection of drug resistance during post-gas shock chemotherapy should also be suppressed, since the emergence of resistance is likely to depend on bacterial population size, which can be reduced rapidly and dramatically by gas treatment. The present work may open a new era of gas-based treatment of tuberculosis and possibly other infectious diseases.

Although treatment of tuberculosis with gas or gas mixture, which is described as an example of the invention disclosed more fully below, serves as a specific embodiment of the present invention, the principles disclosed in the present invention should allow those skilled in the art to extend the application to other disease indications. Thus, as discussed above, the application scope of the present invention is not limited to tuberculosis alone.

Gas or gas mixtures have never been employed alone to treat infectious diseases except for use of hyperbaric oxygen to help cure anaerobic infections. It has been discovered that a variety of gas and gas mixtures can be used to kill Mycobacterium. Passage of an anaerobic gas mixture through cultures of M. tuberculosis (anaerobic shock) causes rapid cell death. While not wishing to be bound by theory, it is thought that (1) hydrogen is the key gas component for extremely rapid and extensive cell death of M. tuberculosis, (2) anaerobic gas mixtures lacking hydrogen kill M. tuberculosis extensively but at a much slower rate than hydrogen or hydrogen-containing gas mixtures, (3) hydrogen-containing gas kills M. tuberculosis whose growth is arrested by a gradual process of oxygen depletion, and (4) hydrogen-oxygen mixtures can kill M. tuberculosis, although at a much slower rate and less extensively than a hydrogen-containing anaerobic gas mixture. Thus, hydrogen and hydrogen-containing gas mixtures can illicit rapid and extensive killing beyond that generally thought to be due to oxygen depletion. Gas-mediated mycobacterial killing is (1) rapid and extensive (e.g., causing more than 7 orders of magnitude reduction in viability in 2-5 min), (2) effective with M. tuberculosis in various physiological conditions (e.g., in growing cultures, in lung homogenates recovered from infected rabbits, and inside human macrophage-like cells), (3) efficacious with MDR and XDR isolates, and (4) non-toxic to human macrophages. Accordingly, Applicants' gas-based approach provides a novel method for treating tuberculosis.

As discussed in the Examples below, several properties of gas-mediated cell death are consistent with gas treatment perturbing an ongoing cellular event that leads to self-destruction by M. tuberculosis: (1) a gas-mediated culture turbidity drop, which, taken as a surrogate of cell death, occurs only with live cells, (2) cell death fails to occur with cells chilled on ice, (3) cell death is insensitive to an inhibitor of protein synthesis, and (4) cell death is specific to M. tuberculosis or M. bovis BCG. Accordingly, Applicants have discovered that hydrogen gas is an active chemical that kills M. tuberculosis rapidly and extensively. Oxygen depletion can facilitate but is not a prerequisite for hydrogen-mediated killing. That leads to three forms of potential clinical applications that directly use gas to treat tuberculosis. The most robust application is to mix a low concentration of hydrogen (e.g., <4%) with air or other gas mixture containing a sufficient amount of oxygen for patients to breathe regularly. A secondary, but more efficacious form of application, involves using oxygenized hydrogen (e.g., <5% oxygen in pure hydrogen or in a hydrogen-inert gas mixture) in a hyperbaric setting to treat patients. In such hyperbaric settings, gas mixtures having very low oxygen concentrations that are not breathable under ambient pressure become directly inhalable. The efficacy of treatment gas should also increase since high pressure and high concentration of hydrogen make it better able penetrate into patient tissues. The most effective way to eliminate tubercle bacilli is to administer hydrogen or a hydrogen-containing anaerobic gas mixture to one lung a time using a double lumen endotracheal intubation. As discussed below, in such methods of treatment one lumen will be connected to the left lung while the other will be connected to right lung. Treatment gas can be pumped into and out of the left lung while oxygen or air will be supplied to the right lung to maintain normal respiration. A switch of gas after a short (e.g., 30 min) treatment will allow both lungs to be treated.

### Composition of Gas Mixture and Direct Inhalation Thereof

One embodiment of the invention relates to a method of treatment of a mycobacterial respiratory tract infection in a patient comprising administering to the patient a safe, hydrogen-containing gas mixture, as described in further detail below, that can be directly inhaled by the patient. Accordingly, in one embodiment the present invention also relates to gas mixtures comprising hydrogen for the treatment of mycobacterial infections. In certain embodiments, the gas mixture comprises sufficient amounts of hydrogen for treatment efficacy of the targeted infection. The gas mixture may further comprise oxygen in sufficient amount for normal respiration so that the gas mixture can be directly inhaled by a patient.

Where the gas mixture is provided at a pressure of about one atmosphere, the gas mixture contains concentrations of oxygen that are high enough to maintain normal respiration, but not so high as to cause hyperoxia toxicity. Accordingly, in certain embodiments the gas mixture comprises oxygen in an amount of from about 15% to about 50% by volume, preferably of from about 17% to about 40% by volume, and more preferably of from about 20% to about 25% by volume. In certain embodiments, the balance of the gas mixture may further comprise an inert or anaerobic gas. In certain embodiments, the inert or anaerobic gas may be selected from the group consisting of nitrogen, helium, argon, carbon dioxide, and mixtures thereof.

In certain embodiments, the gas mixture comprises hydrogen at concentrations that are not explosive when mixed with oxygen sufficient for normal breathing at a pressure of about one atmosphere, which concentrations are readily apparent to one of ordinary skill in the art. Accordingly, in certain embodiments, the gas mixture comprises hydrogen in an amount of about 0.1% to about 4% by volume, preferably of from about 1.0% to about 3.8% by volume, and more preferably of from about 2.5% to about 3.5% by volume. In certain other embodiments, the gas mixture comprises hydrogen in an amount of from about 75% to about 85% by volume, preferably of from about 76% to about 81% by volume, and more preferably of from about 78% to about 80% by volume.

In certain preferred embodiments, the gas mixture at a pressure of about one atmosphere comprises hydrogen in an amount of from about 3% to about 4% hydrogen and oxygen in an amount of from about 21% to about 30% by volume.

The directly breathable gas mixtures can be delivered through a mask from a bag, a compressed cylinder, or in a closed system, such as a inflatable chamber, in which a premixed breathable gas is first used to fill the system, carbon dioxide generated by patient respiration is removed by a carbon dioxide scrubber, and oxygen consumed by the patient is resupplied by a pump through an oxygen source. Hydrogen is not consumed by patients and thus is resupplied only when its concentrations drop below a certain therapeutic target due to accidental leakage.

Method of Treatment Via Intubation with Double Lumen Endotracheal Tube

One embodiment of the present invention relates to a method of treatment of a mycobacterial respiratory tract infection in a patient comprising intubating the patient with a double lumen endotracheal tube, ventilating a first lung infected with the mycobacterial infection with a gas mixture comprising hydrogen, and ventilating a second lung with air or oxygen. Double lumen endotracheal tubes are used for one-lung ventilation in many medical procedures. Double lumen endotracheal tubes are known and commercially available (Covidien, Smiths Medicals, or Med-Worldwide). Typically, a single lumen endotracheal tube is an elongated tube that extends into the trachea of a patient upon intubation and includes one inflatable balloon cuff near its distal end. Commonly, the double lumen endotracheal tube is referred to as an endobronchial tube and, in addition to one lumen which extends to the trachea, has a second longer lumen which extends into the bronchus of a patient upon intubation. Typically, the double lumen endotracheal tube or endobronchial tube includes two inflatable balloon cuffs. These double lumen endotracheal tubes allow for independent control of each lung through the separate lumina. One bronchus may be blocked by occluding one of the lumina at a position external to the patient, in order to isolate a particular lung.

Humans have left and right lungs that can be independently aerated. One lung can be briefly treated with anaerobic gas, while the other can be used to maintain normal respiration. Double lumen endotracheal tubes connected to double-channel respiratory machines (for example, as described in U.S. Pat. No. 4,686,999) are available for such a procedure (Harvard Apparatus). By switching the gas between the lungs, both left and right lungs can be treated. Preliminary data with uninfected rabbits demonstrates that direct treatment can be safely performed. For example, 15 minutes of anaerobic shock with argon to the right lung caused no obvious side-effect.

In certain embodiments, the gas mixture comprises pure hydrogen or a hydrogen-blended anaerobic gas mixture that has no or minimal toxicity to humans. For example, in certain embodiments the gas mixture comprises Bioblend, a gas mixture commercially available from Praxair or GTS-Welco, comprises nitrogen, carbon dioxide, and hydrogen at a ratio of about 85:5:10 percent, respectively. Other gas mixtures containing hydrogen and anaerobic gas, including but not limited to nitrogen, helium, argon, carbon dioxide, and mixtures thereof, can be custom made. In certain other embodiments, the gas mixture comprises nitrogen, argon, and helium at a ratio of about 40:40:20 percent, respectively.

### Method of Treatment Via Hyperbaric Chamber

One embodiment of the present invention relates to treatment of a patient with a hydrogen-containing gas mixture that can be safely inhaled in a hyperbaric setting. Traditional types of hyperbaric chambers are hard shelled pressure vessels that can be run at pressures of up to about six atmospheres. Recent advances in materials technology have resulted in the manufacture of portable, “soft” chambers that can operate at pressures of from about 1.3 to about 1.5 atmospheres. Such devices have been made for breathing high concentrations or high partial pressure of oxygen. The present invention modifies the classical hyperbaric chamber to accommodate direct breathing of low oxygen-high hydrogen gas mixtures that are not breathable at about 1 atmosphere ambient pressure. Since oxygen partial pressure, a product of total absolute pressure and volume fraction of oxygen, determines whether a gas is breathable by humans, a low oxygen volume fraction (e.g., 3%) gas mixture that is not breathable at 1 atmosphere becomes breathable at about 7 atmospheres since the oxygen partial pressure of this gas mixture under such conditions equals to that of ambient air (e.g., about 21% oxygen at 1 atmosphere). Hyperbaric settings are also expected to improve treatment efficacy since at high pressure and concentration, hydrogen, the key component gas for mycobacterial killing, should better able to penetrate patient tissues.

In certain embodiments the method for treatment of a mycobacterial infection in a patient comprises enclosing the patient in a hyperbaric chamber, filling the hyperbaric chamber to a pressure of from about 2 to about 50 atmospheres with a gas mixture comprising hydrogen and oxygen, wherein the oxygen has a partial pressure of about 0.21 (equivalent to that of ambient air), and administering the gas mixture to the respiratory tract of the patient via direct inhalation of the gas mixture. In certain preferred embodiments, the operating pressure in the hyperbaric chamber is of from about 3.5 atmospheres to about 42 atmospheres, more preferably of from about 4.2 atmospheres to about 21 atmospheres, and even more preferably of from about 5 to about 10 atmospheres.

In certain embodiments, the oxygen concentration of the gas mixture in the hyperbaric chamber is less than about 5.3% by volume, preferably of from about 0.4% to about 5% by volume, and more preferably of from about 2.5% to about 4.2% by volume. In certain embodiments in which the gas mixture comprises only hydrogen and oxygen, the oxygen is added to pure hydrogen such that the gas mixture comprises hydrogen in an amount above about 94.7% by volume, preferably of from about 95% to about 99.5% by volume, and more preferably between 95.8% to about 97.5% by volume.

In certain embodiments, oxygen can be added to a hydrogen-anaerobic gas mixture, in which the anaerobic gas is selected from the group consisting of nitrogen, helium, argon, and mixtures thereof. In such embodiments, the gas mixture comprises hydrogen in an amount of from about 1% to about 99% by volume, preferably of from about 4% to about 96% by volume, and more preferably of from about 10% to about 90% by volume.

### Method of Sterilization

Another embodiment of the present discovery relates to a new sterilization method for elimination of infective agents, especially for M. tuberculosis disinfection. Contaminated equipment and environmental surfaces can be treated with hydrogen gas or an anaerobic gas mixture either containing or lacking hydrogen for sterilization without use of harsh chemicals, irradiation, or high temperature that may not be tolerable by the equipment or surface. In certain embodiments, the surface to be sterilized is the skin or body extremity of a patient having a mycobacterial skin infection. In this embodiment the surface to be sterilized with respect to M. tuberculosis would be placed in a chamber, the chamber is vacuumed for about 5-10 minutes, and then hydrogen or a hydrogen-containing anaerobic gas mixture, as described above, is introduced. Treatment time would be about 2-48 hours, preferably about 4-24 hours, and most preferably an overnight (about 16-18 hours) treatment.

### EXAMPLES

The following examples are meant to illustrate, not limit, the scope of the invention.

### Bacterial Species and Growth Conditions

Mycobacterial species, listed in Table 1, were grown at 37° C. in Middlebrook 7H9 or Dubos broth supplemented with 10% ADC, 0.05% Tween 80, and 0.2% glycerol or on 7H10 agar containing the supplements used with 7H9 broth Jacobs, W. R., et al., 1991 Methods Enzymol. 204:537-555. Liquid cultures were grown in 15- or 50-ml tubes using a horizontal roller (Stovall Life Science, Greensboro, N.C.) at 35-40 rpm. Colony formation was detected by growth for 4-8 weeks on 7H10 agar in the presence of 5% CO₂. Escherichia coli, Bacillus sublilis, Shigella flexneri, Salmonella typhimurium, and Pseudomonas aeruginosa were grown in LB broth or on LB agar; Staphylococcus aureus was grown in Mueller-Hinton broth or on Mueller-Hinton agar; Aspergillus fumigatus and Cryptococcus neoformans were grown in YPD (1% yeast extract, 2% peptone, 2% glucose) broth or on YPD agar. All growth was at 37° C. except for Cryptococcus neoformans and Mycobacterium ulcerans, which were grown at 30° C.

TABLE 1
Microbial strains used in the study.

| | Strain | |
|---|---|---|
| Bacterial Species | Number | Relevant Genotype/Phenotype |
| M. tuberculosis | H37Rv | Laboratory strain |
| M. tuberculosis | TN1626 | MDR (Rif^{R}, INH^{R}, Eth^{R}, Kan^{R}, |
| | (KD316) | Str^{R}) |
| M. tuberculosis | KD505 | TN1626 gyrA-r (94G) |
| M. tuberculosis | HN878 | Clinical isolate |
| M. tuberculosis | TN10775 (W4) | 15-INH^{S} |
| M. tuberculosis | TN10536 (KY) | 14-INH^{R} |
| M. tuberculosis | CDC1551 | Clinical isolate |
| M. bovis BCG | Pasteur | Wild type |
| M. fortuitum | ATCC35931 | Human sputum isolate |
| M. xenopi | ATCC19250 | Adult female toad isolate |
| M. smegmatis | mc²155 | Wild type |
| | (KD1163) | |
| M. avium | ATCC25291 | Isolate from diseased hen liver |
| M. marinum | M (ATCC | Clinical isolate |
| | BAA535) | |
| M. ulcerans | ATCC19423 | Clinical isolate |
| Escherichia coli | DM4100 | Laboratory strain (cysB) |
| | (KD65) | |
| Staphylococcus aureus | RN450 | Wild type laboratory strain |
| Pseudomonas | PA01 | Wild type laboratory strain |
| aeruginosa | | |
| Bacillus subtilis | BD630 | Laboratory strain (his, leu, |
| | | met) |
| Salmonella | LT2 (pLM2) | Kan^{R} |
| typhimurium | | |
| Shigella flexneri | 16 (KD276) | Strep^{R}, cold-sensitive |
| Aspergillus fumigantus | MSKCC R21 | Clinical isolate |
| Cryptococcus | H99 | Laboratory reference starin |
| neoformans | | |

### Bacterial Survival Following Anaerobic Shock

Research grade gases, including Bioblend (85% nitrogen, 5% CO₂, and 10% hydrogen), nitrogen, helium, argon, hydrogen, helium-modified Bioblend (85% nitrogen, 10% helium, 5% CO₂), NAH (nitrogen-argon-helium (40%-40%-20%)), hydrogenized air (3.2% hydrogen blended into compressed air), and oxygenized hydrogen (1.5% oxygen mixed with 98.5% hydrogen) were purchased from GTS-Welco Gases Corp (Newark, N.J.). Gases were used to replace ambient air in bacterial cultures by passing the gas through cultures in Vacutainer tubes (BD Medical Supplies, Franklin Lakes, N.J.) at a speed of about 175 mVmin. Compressed air was obtained from a Craftsman compressor. Before and during gas passage, culture aliquots were removed, diluted, applied to agar plates, and incubated as described above. For mycobacteria, plates were incubated for 4-8 weeks for detection of possible delayed growth after anaerobic shock. Bacterial colonies were counted after incubation to determine percent survival relative to colony-forming units (cfu) measured immediately before anaerobic shock.

### Anaerobic Shock of Rabbit Lung Homogenate

Rabbits were infected with M. tuberculosis clinical isolate HN878 via a low-dose aerosol route as previously reported. Sinsimer, D., et al., 2008 Infect Immun 76:3027-36. Briefly, New Zealand white rabbits (˜2.5 kg) were sedated with 0.75 mg/kg acepromazine administered intramuscularly. Each rabbit was placed in a separate, air-tight restraint tube connected to a nasal mask for aerosol delivery. A bacterial suspension (10-15 ml) containing about 10⁷ cfu was placed in the nebulizer cup. Aerosol exposure time was 20 min. At 4 weeks (exponential growth phase) and 8 weeks (chronic, growth-arrest phase) post-infection, rabbits were euthanized with a combination of Ketamine 35 mg/kg and Xylazine 5 mg/kg i.m., followed by Euthasol at 1 ml/10 lbs (4.5 kg) of body weight i.v. Portions of infected lungs lacking the large airways were homogenized in saline (0.9% NaCl, 0.05% Tween 80) using a PRO250 homogenizer (PRO Scientific Inc., Oxford, Conn.). Then samples were placed in Vacutainer tubes, exposed to anaerobic shock as described above for liquid cultures, and plated for cfu determination. The animal work was approved by IACUC of UMDNJ (protocol #07000810).

Anaerobic Shock of Growth-Arrested (Dormant) M tuberculosis Generated by Slow Oxygen Depletion

Cultures of M. tuberculosis CDC 1551 and H37Rv were gradually depleted of oxygen as described by Wayne, L. G., and L. G. Hayes. 1996 Infect Immun 64:2062-9. Briefly, 8.5-ml aliquots of exponentially growing cells were transferred into 13-ml tubes to create a head air volume of 0.5 total tube volume. A sterilized magnetic stirring bar was placed in the bottom of each tube, which was sealed with a sleeved rubber stopper. The tubes were placed in a BIOSTIR digital magnetic stirrer (CAT# W900703, Wheaton Science Products, Millville, N.J.) that was kept inside a 37° C. incubator. After 10, 20, and 30 days of incubation, aliquots were removed for cfu determination before anaerobic shock was directly performed in the original Wayne-model culture tube. After shock viability was determined as described above for rapidly growing cultures.

Anaerobic Shock with Cultured Macrophage-Like Cells Infected with M. tuberculosis

Infection of human macrophage-like cells was performed as described previously Dubnau, E., et al., 2002 Infect Immun 70:2787-95. Briefly, human THP-1 cells were grown in suspension to about 5×10⁵/ml in RPMI 1640 medium containing 10% fetal calf serum. They were then concentrated to about 10⁶ cells/ml by centrifugation and resuspended in fresh medium for treatment with 20 nM phorbol 12-myristate 13-acetate (PMA) for 48 h to induce differentiation. Monolayers of differentiated macrophages were infected with M. tuberculosis H37Rv at an m.o.i. of about 2. Four hours after infection, growth medium was removed, and the monolayer of macrophage-like cells was washed three times with phosphate-buffered saline (PBS) to remove extracellular bacilli. Fresh RPMI 1640 medium was added, and the infected macrophages were incubated for another 44 h. Growth medium was then discarded, and the macrophages were washed with PBS twice before they were trypsinized and concentrated to 5 ml of RPMI medium. Anaerobic shock was performed as with M. tuberculosis cultures. Determination of bacterial viable count was as described above for bacterial cultures except that sodium dodecyl sulfate was added to a final concentration of 0.05% to lyse macrophages following anaerobic shock. M. tuberculosis in macrophage lysates was concentrated by centrifugation, after which cells were washed twice with PBS before dilution and plating on 7H10 agar for determination of percent survival.

### Viability of Human Macrophage-Like Cells Following Anaerobic Shock

THP-1 cells were grown and induced for differentiation as above. The monolayer of differentiated macrophage-like cells was dispersed by trypsinization, after which cell suspensions were transferred to Vacutainer tubes and shocked with anaerobic gas as described for bacterial cultures. At various times, 20-microliter aliquots of suspended cells (˜10⁶ cells/ml) were mixed with an equal volume of Trypan Blue staining solution (0.4% Trypan blue, Sigma Chemicals CO., St. Louis, Mo.)). Total and blue cell numbers were determined by light microscopy using a hemocytometer.

Gas Treatment of M. bovis BCG Growing on Solid Surface

M. bovis BCG cultures were serially diluted and applied onto 7H10 agar plates. Agar plates were placed into anaerobic jars after which the jars were sealed, briefly subjected to a vacuum (2 min), and then flushed with helium (triangles), Bioblend (squares) or hydrogen (circles) for 0, 1, 2, and 4 hour before the plates were taken out of the jars (FIG. 5). After a 4-hour gas flush, one set of jars was sealed for another 20 hours to obtain 24-hour treatment samples. After gas treatment, the plates were incubated at 37° C. for 4-8 weeks in ambient air supplemented with 5% CO₂ for bacterial colony determination. Percent survival, calculated using 0 hour treatment samples as controls, was plotted as a function of treatment time.

Effect of Gases and Gas Mixtures on M. tuberculosis

Abrupt removal of oxygen from the environment causes M. bovis BCG, an organism closely related to M. tuberculosis, to rapidly lyse when an anaerobic gas is rapidly passed through bacterial cultures. Accordingly, the speed of oxygen removal is thought to be important for killing mycobacteria. However, oxygen depletion by passing different anaerobic gas or gas mixtures through M. tuberculosis culture displayed differential effect of killing. Hydrogen turns out to be the key component for rapid and extensive mycobacterial killing since itself or hydrogen-containing anaerobic gas mixtures rapidly and extensively kills M. tuberculosis regardless of its drug-resistance profile and physiological state, and therefore constitutes a novel treatment for tuberculosis and other diseases caused by mycobacteria.

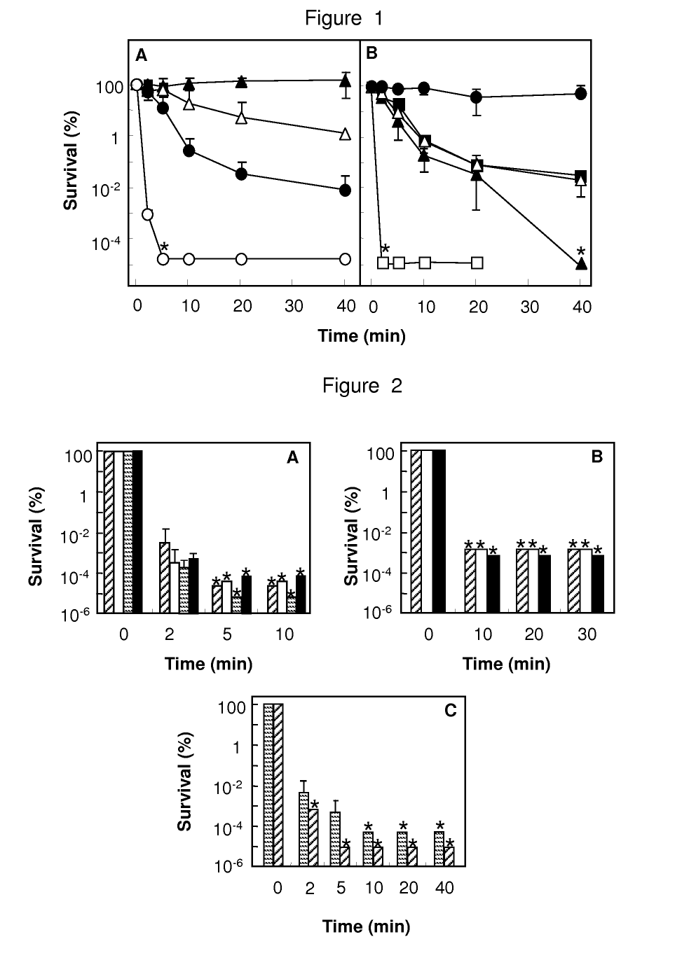

A variety of anaerobic gases were examined for their ability to kill M. tuberculosis, since oxygen depletion has been shown to either cause growth-arrest or cell death of tubercle bacilli. When Bioblend (85% N₂, 10% H₂, and 5% CO₂), an FDA-approved, commercially available anaerobic gas mixture for microbiological testing, was passed through an exponentially growing culture of M. tuberculosis H37Rv, culture turbidity dropped within minutes. Within 2 min after initiating treatment, the viable count dropped 5 orders of magnitude; within 5 min viable count was below the detection limit, dropping from above 10⁸ cfu/ml to below 10 cfu/ml, as illustrated in FIG. 1(A). With respect to FIG. 1, aliquots taken at each time point were serially diluted and applied to 7H10 agar for enumeration of bacterial colonies after 4-8 weeks of incubation at 37° C. Percent survival was plotted as a function of treatment time. In both panels, * indicates that the detection limit (10 cfu/ml) was reached for that time point and thereafter. Error bars indicate standard deviations.

Several gases were examined to better understand Bioblend-mediated bacterial death. Passage of compressed air through M. tuberculosis cultures failed to reduce viability (FIG. 1A). Thus, physical disturbance due to gas passage was not responsible for cell death. Passage of nitrogen, a component of Bioblend, reduced viability by about 10 fold in 5 min and 1,000 fold after 20 min treatment (FIG. 1A). Carbon dioxide, another component of Bioblend, exhibited only a slight lethal effect (FIG. 1A). These data indicate: (I) anaerobic gas-mediated oxygen depletion is not solely responsible for rapid mycobacterial cell death, since drastically different effects were observed with different anaerobic gases; and (2) either the intrinsic feature of Bioblend being a gas mixture or inclusion of hydrogen in Bioblend renders Bioblend superior at killing M. tuberculosis.

To distinguish whether being a gas mixture or hydrogen specifically plays a key role in Bioblend-mediated killing, several additional gases and gas mixtures were examined. The combination of three inert gases (argon, nitrogen, and helium) killed cells more extensively than any of the gases alone, but not as rapidly as Bioblend (FIG. 1B). Thus, treating with a gas mixture per se was not solely responsible for Bioblend-mediated killing. However, replacing hydrogen in Bioblend with helium greatly reduced lethality (FIG. 1B); indeed, hydrogen alone was as effective as Bioblend (FIG. 1B). Thus, hydrogen is the key component for Bioblend-mediated killing. Since in ambient air hydrogen is explosive over a wide range of concentrations and since Bioblend is equally effective, subsequent experiments used Bioblend to avoid safety concerns.

Several experiments were carried out to explore possible mechanisms underlying Bioblend shock-mediated cell death. First, the effect of Bioblend treatment on other microbial species was examined. Killing was specific for M. tuberculosis and its close relative M. bovis BCG, since only these two species, among 16 tested, were killed (Table 2A). Second, M. tuberculosis was treated with Bioblend under various culture conditions. A moderate drop in culture turbidity paralleled viability reduction when live, growing cells were treated (Table 2A), thereby providing a surrogate for killing. No turbidity decrease was observed when cells were heat-killed prior to Bioblend treatment (Table 2B), suggesting that a live cellular event rather than a cell-free chemical or physical reaction is required for Bioblend-mediated killing. Bioblend remained effective when cells were pre-treated with chloramphenicol to block protein synthesis (Table 2B), but gas activity was markedly diminished when M. tuberculosis was treated on ice (Table 2B). Subsequent transfer of samples to 37° C. after treatment on ice led to immediate and extensive cell death (Table 2B). Collectively these data are consistent with Bioblend shock stimulating a cellular component present before shock to trigger rapid and extensive killing.

TABLE 2
Effect of microbial species and M. tuberculosis culture conditions on
Bioblend-mediated cell death.

| | | Culture | |
|---|---|---|---|
| | Strain | turbidity | Viable count |
| | Number | reduction^{a} | reduction^{b} |
| A. Bacterial species | | | |
| Staphyloccus aureus | ATCC | − | − |
| Pseudomonas aeruginosa | PA01 | − | − |
| Bacillus subtilis | BD630 | − | − |
| Escherichia coli | KD65 | − | − |
| Cryptococcus neoformans | H99 | − | − |
| Aspergillus fumigatus | R21 | − | ND^{c} |
| Salmonella typhimurium | LT2 (pLM2) | − | − |
| Shigella flexneri | 16 (KD276) | − | − |
| Mycobacterium avium | ATCC25291 | − | − |
| Mycobacterium fortuitum | ATCC35931 | − | − |
| Mycobacterium xenopi | ATCC19250 | − | − |
| Mycobacterium ulcerans | ATCC19423 | − | − |
| Mycobacterium marinum | M | − | − |
| Mycobacterium smegmatis | mc²155 | − | − |
| Mycobacterium bovis BCG | Pasteur | + | + |
| Mycobacterium | H37Rv | + | + |
| tuberculosis | | | |
| B. M. tuberculosis | | | |
| conditions before/during | | | |
| gas treatment | | | |
| Lethal heat before gas | H37Rv | − | ND^{e} |
| shock^{d} | | | |
| Chloramphenicol before | H37Rv | + | + |
| shock^{f} | | | |
| Chilled with ice during gas | H37Rv | − | − |
| shock | | | |
| Cells shocked on ice for 10 min | H37Rv | + | + |
| and then warmed to | | | |
| 37° C. | | | |

^{a}Culture turbidity was compared before and after a 30-min Bioblend treatment. “−” indicates no change while “+” represents a visual reduction in turbidity.
^{b}Colony forming units after 30 min of Bioblend treatment was compared with untreated control. “−” indicates less than 50% change while “+” represents at least 10-fold reduction.
^{c}Not determined because many filamentous hyphal masses can stick together and appear as a single colony when spread on agar, which makes determination of colony-forming unit on agar an underestimate.
^{d}Exponentially growing cultures were treated at 80° C. for 20 min before exposure to Bioblend.
^{e}Turbidity reduction was used as a surrogate for killing since viable count cannot be determined with cells already killed by heat.
^{f}Exponentially growing cells were treated with 20 μg/mL chloramphenicol for 3 h before exposure to Bioblend.

Effect of Bioblend Shock on Survival of M tuberculosis Strains Differing in Drug Susceptibility and Physiological Status

Two pairs of clinical strains were examined to determine whether Bioblend shock-mediated killing of M. tuberculosis acts with clinical isolates exhibiting various drug-susceptibility profiles. One included an MDR isolate TN1626, which is resistant to rifampicin, isoniazid (INH), ethambutol, kanamycin, and streptomycin, and an isogenic XDR mutant (TN1626-cip) that is also resistant to ciprofloxacin. The second pair included an INH-susceptible (TN 10775) and an INH-resistant isolate (TN 10536) having the same 156110 restriction fragment length polymorphism (RFLP). Death was rapid for all isolates: a 2-min shock reduced viability by at least 4 orders of magnitude, and a slightly longer exposure dropped viable count below the detection limit (e.g. >6 orders of magnitude), as illustrated in FIG. 2(A). With respect to FIG. 2A, exponentially growing cultures of M. tuberculosis were treated with Bioblend for the indicated times. Aliquots taken at each time point were serially diluted and applied to 7H10 agar for enumeration of bacterial colonies after incubation of agar plates at 37° C. for 4-8 weeks; percent survival was expressed as a function of treatment time. In all panels * indicates that the detection limit (10 cfu/ml) was reached; variation in detection limit is due to each isolate having a different bacterial density at the time of treatment. Error bars indicate standard deviations. Thus, these results indicate that Bioblend shock kills both drug-susceptible and drug-resistant M. tuberculosis obtained from clinical sources.

M. tuberculosis taken from infected animals was also examined. Rabbits were infected with M. tuberculosis strain HN878 for 4 weeks (late exponential growth phase) or 8 weeks (chronic, growth-arrest (dormant) phase), lungs were removed and homogenized, and Bioblend was passed through homogenates containing 4 to 7×10⁴ cfu/ml M tuberculosis for 10-30 min. No colony was recovered from gas-treated homogenates from rabbits infected for either 4 or 8 weeks, even at the shortest treatment time, as illustrated in FIG. 2(B). Thus, a clinical isolate of M. tuberculosis, grown in and recovered from rabbit lung, was rapidly killed by Bioblend shock, regardless of whether the bacteria were growing or in a growth-arrest (dormant) state. To confirm that dormant bacteria are rapidly killed, Bioblend was also administered to non-growing persister cells generated by gradual depletion of oxygen. Non-growing and growing bacteria were killed quickly to similar extents, as illustrated in FIG. 2(C).

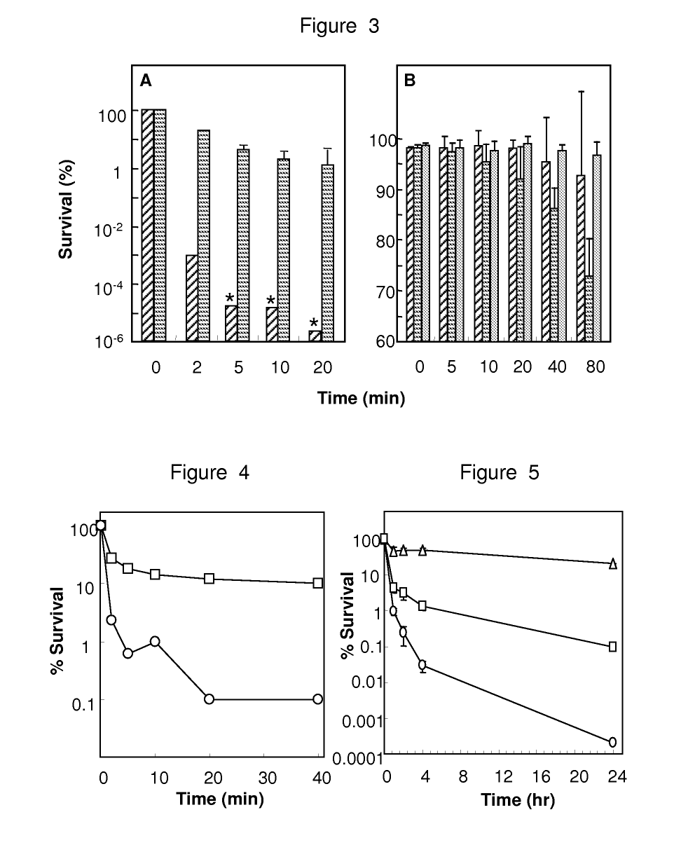

Effect of Bioblend Shock on Survival of M. tuberculosis Inside Human Macrophage-Like Cells M. tuberculosis strain H37Rv was grown inside differentiated THP-1 macrophage-like cells for 2 days, after which the infected cells were treated with Bioblend or argon for the indicated times. THP-1 cells were gently lysed, and the lysate was washed, diluted, and applied to 7H10 agar for enumeration of viable bacterial count. Percent survival was expressed as a function of treatment time. A 2-min Bioblend treatment reduced bacterial viability by 5 orders of magnitude, while a 5-min treatment killed intracellular M. tuberculosis to below the detection limit (e.g. >6 orders of magnitude), as illustrated in FIG. 3(A) (* indicates that the detection limit (10 cfu/ml) was reached; a low detection limit for the 20-min sample is due to an elevated number of cells being plated for viable count at the last treatment point). Consistent with in vitro culture (FIG. 2(B)), argon treatment only reduced bacillary viability moderately (FIG. 3(A)).

The effect of Bioblend on survival of macrophages was also assessed. Human THP-1 cells were induced to differentiate into a monolayer of macrophage-like cells by phorbol 12-myristate 13-acetate (PMA), and then they were recovered as a suspension by trypsin treatment. They were next exposed to Bioblend, argon, or compressed air for various times, and macrophage viability was determined using a trypan blue exclusion assay. Percent of white (live) cells was plotted relative to an untreated sample. Viability was unaffected by either Bioblend or compressed air, as illustrated in FIG. 3(B). Argon slightly reduced viability at long treatment times, as illustrated in FIG. 3(B). These data, along with those in FIG. 3(A), support the idea that a short Bioblend shock kills intracellular M. tuberculosis without harming host cells.

Effect of Hydrogen-Oxygen Mixtures on M. tuberculosis Survival

Since little difference in Bioblend-mediated killing was observed between cells growing aerobically and cells that have been pre-depleted of oxygen from the growth medium for induction of growth arrest (FIG. 2(C)), anaerobiosis may not be a prerequisite for hydrogen-mediated killing. That raises the possibility that hydrogen may be able to kill even in the presence of oxygen. To test this idea, two new, custom-made gas mixtures were prepared that contained oxygen and hydrogen. One blended 3.2% hydrogen into ambient air (hydrogenized air), while the other mixed 1.5% oxygen with 98.5% hydrogen (oxygenized hydrogen). Hydrogenized air killed 90% of cultured M. tuberculosis in 20 min, while oxygenized hydrogen killed 99.9% in the same time period, as illustrated in FIG. 4 (aliquots taken at each time point were serially diluted and applied to 7H10 agar for enumeration of bacterial colonies; percent survival was plotted as a function of treatment time).

These data demonstrate that oxygen inhibits but does not eliminate hydrogen-mediated killing of M. tuberculosis. Since hydrogenized air is directly breathable, it may be used as a robust treatment of pulmonary tuberculosis. Similarly, oxygenized hydrogen, which is not explosive when oxygen concentration is below 5.3%, Dole, M., et al., 1975 Science 190:152-4, can also be directly breathed by patients in a hyperbaric setting (the oxygen partial pressure of a 3% oxygen-97% hydrogen mixture at 7 atmospheres equals that in ambient air at one atmosphere, thereby making such a gas mixture breathable at 7 atmospheres). The high concentration of hydrogen and high pressure in a hyperbaric setting should make hydrogen better able to penetrate lung tissues and thus the hyperbaric setting may greatly increase treatment potency.

Effect of Gas Treatment on Survival of M. bovis BCG Growing on Solid Surface

Killing of mycobacteria growing on a solid surface was also examined. When M. bovis BCG, a close relative of M. tuberculosis, was applied to agar and placed inside a jar that was subsequently flushed with hydrogen or Bioblend, the bacterial cells were killed, as illustrated in FIG. 5. These data indicate that hydrogen or hydrogen-containing anaerobic gas is effective for sterilization of M. tuberculosis-contaminated equipment or environments where toxic and erosive chemicals, irradiation, and high temperature are not suitable. Moreover, the data indicate that skin infections can be treated by gas when caused by mycobacteria that are killed by hydrogen This includes, for example, M. leprae, which is often manifest in body extremities.

What is claimed is:

1. A gas mixture for treatment of a mycobacterial infection comprising hydrogen.

2. The gas mixture of claim 1, further comprising oxygen having a partial pressure of from about 0.17 to about 0.30.

3. The gas mixture of claim 2, further comprising an anaerobic gas.

4. The gas mixture of claim 3, wherein the anaerobic gas is selected from the group consisting of nitrogen, helium, argon, carbon dioxide, and mixtures thereof.

5. The gas mixture of claim 3, wherein the gas mixture at about one atmosphere of pressure comprises hydrogen in an amount of from about 0.1% to about 85% by volume.

6. The gas mixture of claim 3, wherein the gas mixture at about one atmosphere of pressure comprises hydrogen in an amount of from about 1.0% to about 83% by volume.

7. The gas mixture of claim 3, wherein the gas mixture at about one atmosphere of pressure comprises hydrogen in an amount of from about 2.5% to about 3.5% by volume or about 78% to about 80% by volume.

8. A method for treatment of a mycobacterial respiratory tract infection in a patient comprising administering a gas mixture comprising hydrogen and oxygen to the respiratory tract of the patient via direct inhalation at a pressure of about 1 atmosphere.

9. The method of claim 8, wherein the gas mixture further comprises an anaerobic gas.

10. The method of claim 9, wherein the anaerobicgas is selected from the group consisting of nitrogen, helium, argon, and mixtures thereof.

11. The method of claim 8, wherein the mycobacterial infection is an infection of M. tuberculosis.

12. The method of claim 8, wherein the gas mixture comprises hydrogen in an amount of from about 0.1% to about 4% by volume or about 75% to about 85% by volume, and wherein the gas mixture comprises oxygen in an amount of from about 15% to about 50% by volume.

13. The method of claim 8, wherein the gas mixture comprises hydrogen in an amount of from about 1.0% to about 3.8% by volume or about 76% to about 81% by volume, and wherein the gas mixture comprises oxygen in an amount of from about 17% to about 40% by volume.

14. The method of claim 8, wherein the gas mixture comprises hydrogen in an amount of from about 2.5% to about 3.5% by volume or about 78% to about 80% by volume, and wherein the gas mixture comprises oxygen in an amount of from about 20% to about 25% by volume.

15. A method for treatment of a mycobacterial respiratory tract infection in a patient comprising:
(a) intubating the patient with a double lumen endotracheal tube;
(b) ventilating a first lung containing the mycobacterial infection with a gas mixture comprising an anaerobic gas; and
(c) ventilating a second lung with air or oxygen.

16. The method of claim 15, wherein the anaerobic gas is selected from the group consisting of hydrogen, nitrogen, argon, helium, carbon dioxide, and mixtures thereof.

17. The method of claim 16, wherein the gas mixture at a pressure of about one atmosphere comprises:
(a) hydrogen in an amount of about 10% by volume;
(b) nitrogen in amount of about 85% by volume; and
(c) carbon dioxide in an amount of about 5% by volume.

18. The method of claim 15, wherein the anaerobic gas is selected from the group consisting of nitrogen, argon, helium, carbon dioxide, and mixtures thereof.

19. The method of claim 18, wherein the gas mixture at a pressure of about one atmosphere comprises:
(a) nitrogen in an amount of about 40% by volume;
(b) argon in amount of about 40% by volume; and
(c) helium in an amount of about 20% by volume.

20. A method for treatment of a mycobacterial respiratory tract infection in a patient comprising:
(a) enclosing the patient in a hyperbaric chamber;
(b) filling the hyberbaric chamber to a pressure of from about 3.5 to about 50 atmospheres with a gas mixture comprising hydrogen and oxygen, wherein the oxygen has a partial pressure of from about 0.17 to about 0.30; and
(c) administering the gas mixture to the respiratory tract of the patient via direct inhalation of the gas mixture.

21. The method of claim 20, wherein the pressure in the hyberbaric chamber is from about 4 to about 10 atmospheres.

22. The method of claim 20, wherein the gas mixture further comprises an anaerobic gas selected from the group consisting of nitrogen, helium, argon, and mixtures thereof.

23. A method for the sterilization of mycobacterium-contaminated surfaces comprising exposing the surface to a gas mixture comprising hydrogen.

24. The method of claim 23, wherein the surface is the skin of a patient having a mycobacterial skin infection.
